(19) United States
(12) Patent Application Publication
(10) Pub. No.: US 2007/0167720 A1
(43) Pub. Date: Jul. 19, 2007

(54) Smart card control of medical devices

(76) Inventors: Raju R. Viswanathan, St. Louis, MO (US); Bevil J. Hogg, St. Louis, MO (US); John Kinder, St. Louis, MO (US); David Olson, St. Louis, MO (US)

Correspondence Address: Bryan K. Wheelock, Suite 400, 7700 Bonhomme, St. Louis, MO, 63105, US

(21) Appl. No.: 11/634,826

(22) Filed: Dec. 6, 2006

Related U.S. Application Data
(60) Provisional application No. 60/742,850, filed on Dec. 6, 2005.

(51) Int. Cl. A61B 5/05

(52) U.S. Cl. 600/407

(57) ABSTRACT

A method of exploiting a medical technology comprising: providing at least two types of medical systems incorporating the medical technology. Each of the systems has first capabilities useful in performing a first type of medical procedure, and second capabilities useful in performing a second type of medical procedure. One of the medical systems is offered in exchange for payment allowing access to both the first and second capabilities, and the other of the medical system is offered will full access to the first capabilities in exchange for payment, while access to the second capabilities are restricted and separately sold to the purchaser on an episodic basis.

### CROSS-REFERENCE TO RELATED APPLICATIONS

This application claims the benefit of U.S. Provisional Patent Application Ser. No. 60/742,850, filed Dec. 6, 2005, the entire disclosure of which is incorporated herein by reference.

### BACKGROUND

The invention relates to methods of commercializing medical technology, and to medical systems specifically adapted for commercialization.

There are economic challenges introducing new medical technologies. Healthcare providers hesitate to invest in new devices without a proven track record and known return on investment. Medical device companies make substantial investments in developing a new technology, proving the new technology, obtaining the necessary approvals and market the technology. New device companies have to balance device features with price point. Niche applications develop for the new technology, for which the company has to decide between developing cheaper, specialized devices for the niche market or attempting to sell the full featured devices for the niche application. The full-featured system may be too expensive for widespread use in the niche application, and less-expensive specialized devices may cannibalize sales of the full-featured system.

### SUMMARY

Generally, embodiments of the present invention improve the distribution and acceptance of new medical technology by providing specialized versions of medical systems targeted for niche markets at potentially lower cost, which do not cannibalize sales of larger, full featured systems. This allows a new technology to more quickly gain acceptance in niche markets and demonstrate the utility and cost effectiveness of the overall technology.

In accordance with one embodiment of the methods of this invention, a method of exploiting a medical technology is provided in which at least two types medical systems incorporating the medical technology are provided, each system having first capabilities useful in performing a first type of medical procedure, and second capabilities useful in performing a second type of medical procedure. One of these systems is offered for sale or lease in which access to both the first and second capabilities is allowed. The other of these systems is offered for sale or lease in which access to the second capabilities are restricted, and access to the second capabilities of the system are sold separately on an episodic basis.

In accordance with another embodiment of the methods of this invention, a method of commercializing a medical system having first capabilities useful in performing a first type of medical procedure, and second capabilities useful in performing a second type of medical procedure, is provided. In this embodiment the medical system is sold or leased and access to the second capabilities of the system is restricted. Access to the second capabilities of the medical system are sold separately on an episodic basis.

Access to the second capabilities can be sold on a per procedure basis, or a per unit time basis. The access can be implemented, using tokens, such as an access card, which can be a single use, multiple use, or replenishable card. The apparatus and methods can be adapted to any medical systems, including those which already employ some type of electronic or smart card, for example those that require use of a smart card to enable use with a medical device.

In accordance with another embodiment of this invention, a medical system is provided having first capabilities useful in performing a first type of medical procedure, and second capabilities useful in performing a second type of medical procedure. The first capabilities are available to all users, and the second capabilities are available only to users with a token which enables the second capabilities of the medical system. The access can be implemented, for example, using tokens, such as an access card, which can be a single use, multiple use, or replenishable card.

In accordance with another embodiment of this invention, a method of controlling access to a medical system having first capabilities useful in performing a first type of medical procedure, and second capabilities useful in performing a second type of medical procedure is provided comprising restricting access to the second capabilities to users with a procedure token. The token can be, for example, an access card, which can be a single use, multiple use, or replenishable card. The apparatus and methods can be adapted to any medical systems, including those which already employ some type of smart card, for example those that require use of a smart card to enable use with a medical device.

Thus various embodiments provide for the marketing of medical systems with restricted capabilities which can be offered at lower initial cost that full featured systems, which establish a larger installed base, help demonstrate the capabilities and value of the technology, yet do not cannibalize sales of more full featured systems. These embodiments also allow restricting selected capabilities of a medical system to those with appropriate training or authorization. These and other features and advantages will be in part apparent, and in part pointed out hereinafter.

### BRIEF DESCRIPTION OF THE DRAWINGS

FIG. 1A is a schematic diagram of a medical system implementing one preferred embodiment of this invention;

FIG. 1B is a schematic diagram of a medical system implementing an alternate of the preferred embodiment of this invention.

FIG. 2A is a flow chart of one possible method of operating the system of FIG. 1A and

FIG. 2B is a flow chart of one possible method of operating the system of FIG. 1B.

Corresponding reference numerals indicate corresponding parts throughout the several views of the drawings.

### DETAILED DESCRIPTION

Various embodiments of this invention provide for the improved commercializing and/or enhanced security for medical systems. According to a first embodiment, a method of exploiting a medical technology is provided in which at least two types of medical systems incorporating the medical technology are marketed. Each system has first capabilities useful in performing a first type of medical procedure, and second capabilities useful in performing a second type of medical procedure. The first system provides full access to the first and second capabilities. The second system has access to the second capabilities restricted. Both systems are offered for sale or lease, purchasers or leasees of the first system have full access to the first and second capabilities. However, purchasers or leasees of the second system only have access to the first capabilities, and have to purchase access to the second capabilities if they want them on an episodic basis.

For example, a general purpose magnetic navigation system for magnetically navigating medical devices through the body has been developed. This full featured system can be used to orient medical devices in any direction in the body. The system includes a user interface that allows the physician to direct the navigation in a number of different ways, appropriate for a variety of different medical procedures. The system is relatively expensive, and upon initial introduction would generally be considered only by first-tier hospitals. However, magnetic navigation is particularly advantageous for a number of special applications that might not justify the cost of a full featured system. However, reducing to the price of the full featured system to capture these niche applications would eliminate sales at full price and prevent the developer from recouping its development costs. However, a specialized system for the particular application can be developed and sold. To the extent that this specialized system inherently has similar capabilities to the full featured system that were not necessary for the niche application, there is a risk that healthcare providers would purchase the specialized system for broader use, and not by the full featured system. By restricting access to features, the specialized system can be sold without risk to the full featured system, allowing penetration of the technology. By separately selling access to the restricted features, the installed base of potential users is much larger than the number of full featured systems sold. Thus, a wider audience has exposure to the technology, promoting the technology beyond the niche applications.

In accordance with another embodiment of this invention, a method of commercializing a medical system is provided. The system has first capabilities useful in performing a first type of medical procedure, and second capabilities useful in performing a second type of medical procedure. The system is sold or leased to users with full access to the first capabilities. However, access to the second capabilities are restricted, and access to these second capabilities are sold to purchaser/leasee on an episodic basis. This method allows a relatively inexpensive system to be distributed for fast penetration for niche applications, and serve as a marketing base for the additional capabilities of the technology.

The second capabilities can be sold on a per procedure basis or a per unit time basis. This can be conveniently done by selling a token which causes the medical system to permit access to the systems second capabilities. This token is preferably in the form of a smart card, with a magnetic, electronic, or optical memory, but could be in some other form. The smart card has a memory in which an access code is stored and can be read (and preferably also written to) by the medical system. Codes can be developed enabling access to the second capabilities for an entire medical procedure, or for a predetermined period of time (which is selected to cover an entire procedure with a margin of safety). Cards with a single procedure or a single period of time, or a plurality of procedures or plurality of periods of time can be sold. These cards can preferably can be replenished, either by returning them to the vendor, or on-line. In the latter regard, the medical systems are preferably connected to the internet, and can be updated with security information so that the cards function properly.

An additional benefit of this method is that is provides security, restricting access to some of the capabilities of the system to persons who have the expertise and/or training to use those features.

Systems have already been developed in which operation of a medical system with a particular medical device is dependent upon a device token associated with the device. Such a system is disclosed in U.S. patent application Ser. No. 10/674,914, filed Sep. 30, 2003, based on U.S. Provisional Patent Application Ser. No. 60/414,574, filed Sep. 30, 2002, entitled A Method and Apparatus for Improved Surgical Navigation Employing Electronic Identification with Automatically Actuated Flexible Medical Devices, the disclosure of which is incorporated herein by reference. The methods of the present invention can be adapted for use with such systems, with a device token required to operate the system, and a procedure token required to enable selected capabilities of the system. Thus, only one set of devices and device tokens has to be manufactured and marketed, simplifying inventory and distribution. The token can be electronic, magnetic, optical or can incorporate other means of data storage familiar to those skilled in the art.

In still another embodiment of this invention, a medical system is provided having first capabilities useful in performing a first type of medical procedure, and second capabilities useful in performing a second type of medical procedure. The first capabilities are available to all users, with the second capabilities being available only to users with a token which enables the second capabilities of the medical system. This token is preferably in the form of a smart card, with a magnetic, electronic, or optical memory, but could be in some other form. The smart card has a memory in which an access code is stored and can be read (and preferably also written to) by the medical system. Codes enabling access to the second capabilities for an entire medical procedure, or for a predetermined period of time (which is selected to cover an entire procedure with a margin of safety). Cards with a single procedure or a single period of time, or a plurality of procedures or plurality of periods of time can be sold. These cards can preferably can be replenished, either by returning them to the vendor, or on-line. In the latter regard, the medical systems are preferably connected to the internet, and can be updated with security information so that the cards function properly.

As discussed above, systems have already been developed in which operation of a medical system with a particular medical device is dependent upon a device token associated with the device. Such a system is disclosed in U.S. patent application Ser. No. 10/674,914, filed Sep. 30, 2003, based on U.S. Provisional Patent Application Ser. No. 60/414,574, filed Sep. 30, 2002, entitled A Method and Apparatus for Improved Surgical Navigation Employing Electronic Identification with Automatically Actuated Flexible Medical Devices, the disclosure of which is incorporated herein by reference. The systems of the present invention can be adapted for use with such systems, with a device token required to operate the system, and a procedure token required to enable selected capabilities of the system. Thus, only one set of devices and device tokens has to be manufactured and marketed, simplifying inventory and distribution. In an alternate embodiment, both procedure and device enablement can be available with a single token. The form or content or both of the User Interface which is used to control the system can also depend on the information supplied by the token, in order to correspond to the desired procedure type.

The system preferably includes a control which can read and preferably also write to the token. The control reads access information from the card and determines whether the access information is valid. After the access information is used, the control preferably writes this information to the card. Alternatively the control can read identification information, and check the access information relative to a database, preferably via the internet, to determine the validity of the card

In yet another embodiment of this invention, a method is provided for controlling access to a medical system having first capabilities useful in performing a first type of medical procedure, and second capabilities useful in performing a second type of medical procedure. Access to the second capabilities are restricted to users with a procedure token.

As discussed above, systems have already been developed in which operation of a medical system with a particular medical device is dependent upon a device token associated with the device. Such a system is disclosed in U.S. patent application Ser. No. 10/674,914, filed Sep. 30, 2003, based on U.S. Provisional Patent Application Ser. No. 60/414,574, filed Sep. 30, 2002, entitled A Method and Apparatus for Improved Surgical Navigation Employing Electronic Identification with Automatically Actuated Flexible Medical Devices, the disclosure of which is incorporated herein by reference. The systems of the present invention can be adapted for use with such systems, with a device token required to operate the system, and a procedure token required to enable selected capabilities of the system. Thus, only one set of devices and device tokens has to be manufactured and marketed, simplifying inventory and distribution. In an alternate embodiment, both procedure and device enablement can be available with a single token. The form or content or both of the User Interface which is used to control the system can also depend on the information supplied by the token, in order to correspond to the desired procedure type.

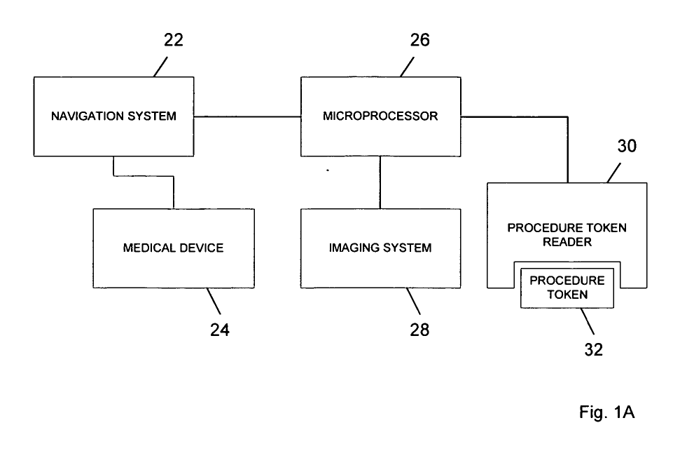

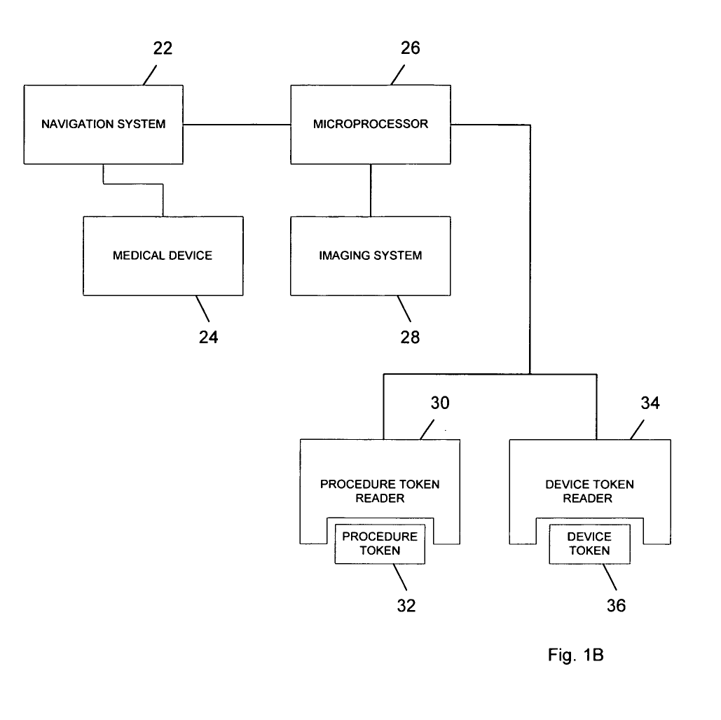

One embodiment of a system in accordance with the principles of this invention is indicated generally as 20 in FIG. 1A. As shown in and described, system 20 is a remote navigation system for navigating a medical device in the body, such as a magnetic navigation system available from Stereotaxis, Inc., St. Louis, Mo. However, the invention is not so limited, and could be employed with any medical system, including diagnostic and therapeutic systems, such as medical imaging systems. shown schematically in FIG. 1A.

The system 20 comprises a magnetic navigation system 22, which is adapted for remotely navigating a medical device 24 through a subject's body. The system may include one or more external magnetics for creating a magnetic field in an operating region inside the subject's body in a selected direction to orient the distal end of the medical device 24, which is provided with one or more magnetically responsive elements. Once the distal end of the medical device is oriented in the desired direction, it can be advanced by advancing the proximal end of the device.

The system 20 includes an imaging system 28, which may be an x-ray, ultrasound, MR, or other medical imaging system, for providing images of the operating region inside the subject. The system 20 also includes a computer 30 for controlling the navigation system 22 and the imaging system 28.

In accordance with principles of this invention, the system 20 also includes a procedure token reader 30 adapted for use with one or more procedure tokens 32. The computer 26 controls the access to the magnetic navigation system 22 and the imaging system 28, allowing the navigation system 22 and imaging system 28 to be used for some medical procedures, but restricting the system from being used for other medical procedures. For example the computer may for example, restrict use during certain dates or times, or with certain medical devices 24, or in conducting particular types of medical procedures. However the computer may permit the system to be used despite these restrictions, if the user presents an appropriate token authorizing the procedure. The system includes a procedure token reader 30 that is adapted to read or otherwise obtain authorization information from a procedure token 32. The reader and token could comprise any system for reading access information, for example the procedure token could have printed bar code information, and the procedure token reader could be a bar code reader; the procedure token could have an rf-id circuit, and the procedure token reader could be rf circuit reader; the procedure token could have an embedded memory chip, and the procedure token reader could be a memory chip reader. Of course rather than a physical token, the procedure token could be a signal that is received by the procedure token reader, for example a signal or code received via the internet or cellular device.

The system 20 can be used for any of the permitted procedures, and when it is desired to use the system 20 for a restricted procedure, the user simply provides the appropriate procedure token 32 to the procedure token reader 30.

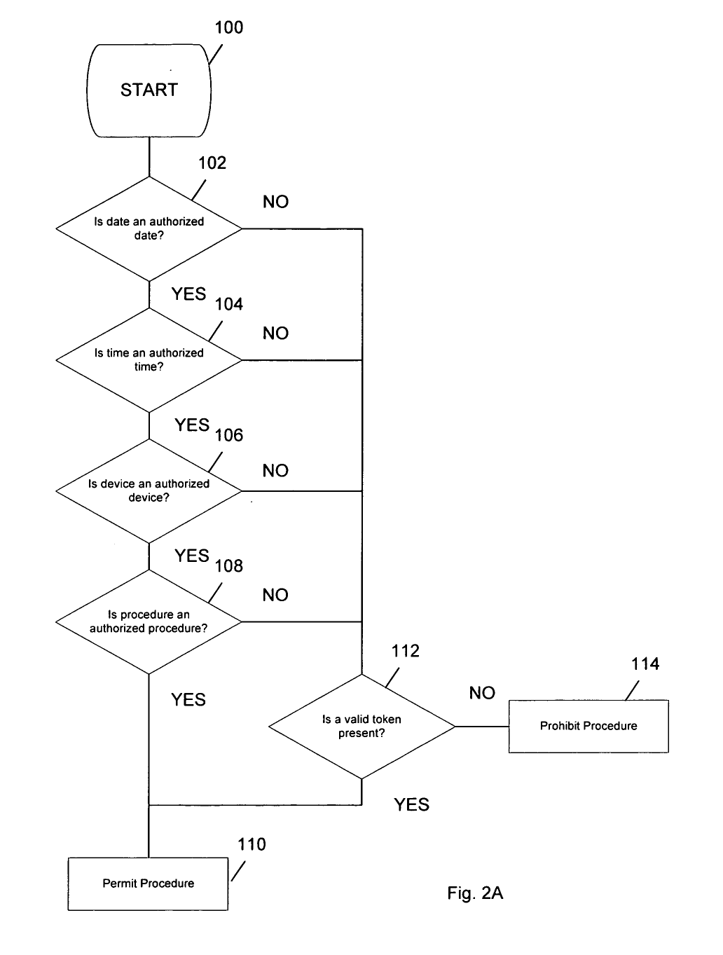

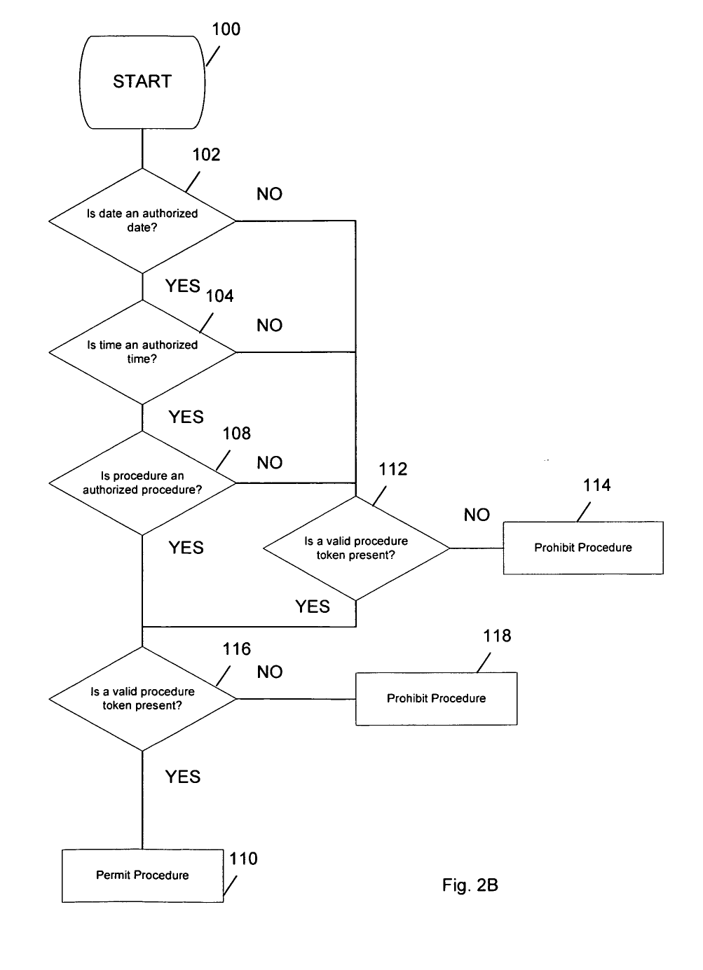

A possible method of operation of the system 20 is shown in FIG. 2A. As shown in FIG. 2A, the procedure starts at 100. At step 102 the computer determines whether the current date is a date for which use of the navigation system is not restricted. At step 104 the computer determines whether the current time is a time for which the use of the navigation system is restricted. At step 106, the computer determines whether the device being used with the navigation system is a device for which the use of the navigation system is restricted. Finally, at step 108, the computer determines whether the procedure for which the navigation system will be used is a procedure for which the navigation system is restricted. If no restrictions apply, then at 110 the navigation system is permitted to operate. At 112, if any of the restrictions apply, then the system determines whether an appropriate procedure token is present, if the appropriate procedure token is not present, then at 110 navigation system is permitted to operate. If the appropriate procedure token is not present, then at 114 the navigation system is not permitted to operate. Of course there could be fewer, more, or different restrictions placed on the operation of the navigation system, and there is no particular importance to the order of the steps.

An alternate construction of the system of FIG. 1A is indicated generally as 20′ in FIG. 1B. The system 20′ is similar to system 20, and corresponding parts are identified with corresponding reference numerals, but system 20′ also comprises a device token reader 34 for use with a device token 36. This allows the system 20′ to lock out the navigation system 22 from operating with a particular medical device 24 unless a corresponding device token 36 is provided. This prevents the system 22 from being used with unauthorized devices, and can also be used to program the computer 26 controlling the navigation system 22 with information about the particular device 24, so that the operation of the navigation system is adapted for the properties of the particular device. Of course rather than a separate procedure token reader 30 and a device token reader 34, a single token reader could be provided. Similarly, rather than a separate procedure token 32 and device token 36, a single token could be provided.

A possible method of operation of the system 20′ is shown in FIG. 2B. The method shown in FIG. 2B is similar to the method of operation of the system 20 shown in FIG. 2A, and corresponding steps are identified with corresponding reference numerals. As shown in FIG. 2B, the procedure starts at 100. At step 102 the computer determines whether the current date is a date for which use of the navigation system is not restricted. At step 104 the computer determines whether the current time is a time for which the use of the navigation system is restricted. Finally, at step 108, the computer determines whether the procedure for which the navigation system will be used is a procedure for which the navigation system is restricted. If no restrictions apply, then operation of the system is not restricted. At 112, if any of the restrictions apply, then the system determines whether an appropriate procedure token is present, if the appropriate procedure token is not present, then operation of the system is not restricted. If the appropriate procedure token is not present, then at 114 the navigation system is not permitted to operate. Of course there could be fewer, more, or different restrictions placed on the operation of the navigation system. Then at step 116 the system determines whether the appropriate device token is present for the device that is being used for the procedure. If the appropriate device token is present, then at 110 the navigation system is permitted to operate. If the appropriate device token is not present, then at 118 the navigation system is not permitted to operate.

What is claimed is:

1. A method of exploiting a medical technology comprising: providing at least two types of medical systems incorporating the medical technology, each system having first capabilities useful in performing a first type of medical procedure, and second capabilities useful in performing a second type of medical procedure; offering to transfer one type of medical system in exchange for payment allowing access to both the first and second capabilities or to transfer the other type of medical system in exchange for payment, while restricting access to the second capabilities of the system, and separately selling access to the second capabilities of the medical system on an episodic basis.

2. A method of commercializing a medical system having first capabilities useful in performing a first type of medical procedure, and second capabilities useful in performing a second type of medical procedure, the method comprising:
transferring the medical system to a user in exchange for payment, while restricting access to the second capabilities of the system, and separately selling access to the second capabilities of the medical system on an episodic basis.

3. The method according to claim 1 wherein access to the second capabilities are sold on a per procedure basis.

4. The method according to claim 1 wherein access to the second capabilities are sold on a per unit time basis.

5. The method according to claim 1 wherein access is restricted to predetermined periods on a calendar.

6. The method according to claim 1 wherein access to the second capabilities are delivered by providing a token which causes the medical system to permit access to the systems second capabilities.

7. The method according to claim 4 wherein the token provides access to the second capabilities for a single procedure.

8. The method according to claim 4 wherein a user can replenish the token.

9. The method according to claim 4 wherein the token provides access to the second capabilities for a plurality of procedures.

10. The method according to claim 9 wherein the user can replenish the token.

11. The method according to claim 4 wherein the token provides access to the second capabilities for a predetermined period of time.

12. The method according to claim 1 wherein the system is adapted for use with a medical device, and wherein operation of the system is dependent upon a token associated with the medical device.

13. A medical system having first capabilities useful in performing a first type of medical procedure, and second capabilities useful in performing a second type of medical procedure, the first capabilities being available to all users, and the second capabilities being available only to users with a token which enables the second capabilities of the medical system.

14. The medical system according to claim 13 wherein the token provides access to the second capabilities for a single procedure.

15. The medical system according to claim 13 wherein the token provides access to the second capabilities for a predetermined period of time.

16. The system according to claim 1 wherein the wherein the system is adapted for use with a medical device, and wherein operation of the system is dependent upon a token associated with the medical device.

17. A method of controlling access to a medical system having first capabilities useful in performing a first type of medical procedure, and second capabilities useful in performing a second type of medical procedure, the method comprising restricting access to the second capabilities to users with a procedure token.

18. The method according to claim 17 wherein the medical system is adapted for use with a medical device, the method further comprises restricting operation of the system without a device token associated with the device.

19. A medical system having first capabilities useful in performing a first type of medical procedure, and second capabilities useful in performing a second type of medical procedure, the system including a control for restricting access to the second capabilities to users with a procedure token.

20. The medical system of claim 19 where the token is an electronic smart card.

21. The medical system according to claim 19 wherein the procedure token provides access to the second capabilities for a single procedure.

22. The medical system according to claim 19 wherein the procedure token provides access to the second capabilities for at least one predetermined period of time.

23. The method according to claim 19 wherein the medical system is adapted for use with a medical device, the method further comprises restricting operation of the system without a device token associated with the device.

24. A medical system having first capabilities useful in performing a first type of medical procedure, and second capabilities useful in performing a second type of medical procedure, the system including a control for restricting access to the second capabilities to users with a procedure token, based on which a corresponding User Interface is displayed to users of the medical system.

25. The medical system according to claim 24 wherein the procedure token provides access to the second capabilities for at least one predetermined period of time.
